(19) United States
(12) Patent Application Publication
(10) Pub. No.: US 2024/0296950 A1
(43) Pub. Date: Sep. 5, 2024

(54) Remote Monitoring Server and Method of Operating Same

(71) Applicant: BIOTRONIK SE & Co. KG, Berlin (DE)

(72) Inventors: Larry STOTTS, Fulshear, TX (US); Lauren KRAITER, Tigard, OR (US); James HORTON, Portland, OR (US); Marko TIETZ, Berlin (DE)

(73) Assignee: BIOTRONIK SE & Co. KG, Berlin (DE)

(21) Appl. No.: 18/551,441

(22) PCT Filed: Mar. 14, 2022
(86) PCT No.: PCT/EP2022/056423

§ 371 Date: Sep. 20, 2023

Related U.S. Application Data
(60) Provisional application No. 63/167,706, filed on Mar. 30, 2021.

(30) Foreign Application Priority Data
May 17, 2021 (EP) 21173998.2

(51) Int. Cl. G16H 50/20; G16H 15/00; G16H 40/67

(52) U.S. Cl. CPC G16H 50/20; G16H 15/00; G16H 40/67

(57) ABSTRACT

A remote monitoring server (RMS) configured to be used in a system comprising a medical device and a remote device of the group comprising a patient remote device and a health care professional remote device. The RMS comprises a communication module configured to communicate with the remote device, a central data repository, and a processor, wherein the central data repository is configured to store data collected by the medical device and the remote device, wherein the RMS further comprises an artificial intelligence (AI) module integrated within the processor and/or cooperating with the processor, wherein the AI module is configured to process data stored in the central data repository and/or to control data communication using the communication module and/or to provide services of a search engine. Further provided is a system comprising the RMS, the medical device and the remote device, and a method for operating the RMS.

### CROSS-REFERENCE TO RELATED APPLICATIONS

This application is the United States National Phase under 35 U.S.C. § 371 of PCT International Patent Application No. PCT/EP2022/056423, filed on Mar. 14, 2022, which claims the benefit of European Patent Application No. 21173998.2, filed on May 17, 2021 and U.S. Provisional Patent Application No. 63/167,706, filed on Mar. 30, 2021, the disclosures of which are hereby incorporated by reference herein in their entireties.

### TECHNICAL FIELD

This invention disclosure describes a remote monitoring server configured to be used in a system comprising at least one medical device and at least one remote device of the group comprising at least one patient remote device and at least one health care professional (HCP) remote device and a method of operating same. The present invention further describes a computer program product and a computer readable data carrier.

### BACKGROUND

Medical devices such as implants as known in the art can be coupled with various external (remote) devices to exchange data. For example, an implantable medical device can be coupled with a programmer that allows a health care professional (HCP), such as a clinician, to adjust the implantable medical device's settings. For coupling a magnetic switch can, for example, be used, which is placed over the implantable medical device. The magnetic switch has a permanent magnet whose magnetic field is detected by the implantable medical device and puts it into a coupling state in which the implantable medical device is coupled to the programmer.

In addition, in a wireless monitoring system, a patient device is known to connect wirelessly to a medical device when located near the medical device. Such patient devices are used, for example, to download patient data at regular intervals (e.g., daily) from the implantable medical device and forward it to a service centre. At the service centre, the data are pre-processed and, if necessary, transferred to a user, e.g., an HCP.

A patient device may, for example, be equipped with a wake-up function to switch an associated implantable medical device from a resting state to an active state in order to establish a communication link with the implantable medical device. The patient device, for example, emits a wake-up signal in a radio frequency (RF) range, which is received by the implantable medical device, whereupon it is activated for data communication with the patient device.

A potential disadvantage of a general wake-up functionality to wake up an implantable medical device is that unwanted access to the implantable medical device can potentially be gained by third-party devices that implement the patient device's communication scheme and methodology. Especially, in the field of implantable medical devices with vital therapy functions such as pacemakers and implantable cardioverter defibrillators, demands on cybersecurity are generally high.

In addition, systems exist providing the possibility for a patient to initiate a recording of data by the implantable medical device. If, for example, a pacemaker patient does not feel well, e.g., if the patient feels dizzy or experiences a tachycardia, the patient can trigger the recording of an ECG by the implantable medical device, e.g., by actuating a remote control. The data recorded in this way is transferred directly to an HCP or to a service centre the next time data is retrieved from the implantable medical device, for example during an examination at the clinician or via an automatic query of the patient device.

In a typical workflow, as provided by some products currently available, a patient, who feels symptomatic, may wish to send a report to a clinic, or the clinic may request that the patient generate a report. In either case, the patient follows the process to initiate the patient device to trigger the interrogation. This requires either that the patient places an interrogation wand over the implanted medical device, or if available, the patient device can connect to the implanted medical device via wireless means. The implanted medical device processes the interrogation and transmits data to the patient device. The patient then triggers the patient device to transmit an interrogation message, for example, to a service centre, or if available, the patient device initiates the transmission to the service centre automatically.

Thus, such solutions are patient-triggered in the sense that clinicians must rely on the patient to initiate the data capture and possibly the data transmission as well, and are therefore not in control of how or when the data is captured. Further, as the clinic cannot control receipt of the information transmitted from the implant, an HCP is not necessarily able to respond to the receipt of the data at the time it is received. This leads to a non-optimal workflow regarding the clinic review of the data. Because the transmission of data triggered by the patient and the receipt and review by the clinic are disconnected from one another, potentially a liability situation may be caused, if the clinic does not respond in a timely fashion.

State-of-the-art implanted pacemakers, cardiac monitors, neuro stimulators and similar implantable medical devices communicate with an HCP user interface during a face-to-face visit with an HCP. During the face-to-face visit the programmer provides a real-time view of diagnostic and technical data. These implanted devices can also typically transmit diagnostic and technical data periodically to a server. HCPs may access this data through a secure website.

However, the user interface of such website provided for the HCPs to interact with this data typically provides no other service than a display of the patient IMD interrogation report collected in-office and/or via remote monitoring. The reports available are frequently too limiting for HCPs to provide more complex care to their patients or for use of this data in other aspects of the clinical practice management.

Accordingly, there is a need for a remote monitoring server and a system comprising this server which improves medical and related data management and processing as well as provides a more individual and broader usage of the data including data communication, for example, not only for the patient him-/herself and his/her personal medical device but also for quality management and general operation's control of medical devices, clinical studies, analyses for clinic's business control, etc. Further, clinic workflows need to be optimized in order to reduce costs.

The present disclosure is directed toward overcoming one or more of the above-mentioned problems, though not necessarily limited to embodiments that do.

### SUMMARY

At least the above object is solved by a remote monitoring server having the features of claim 1, a system comprising this server with the features of claim 7 and a method with the features defined in claim 9. Further, at least the object is solved by a computer program product with the features of claim 14 and a computer readable data carrier having the features of claim 15.

Particularly, at least the above object is solved by a remote monitoring server (RMS) configured to be used in a system comprising at least one medical device and at least one remote device of the group comprising at least one patient remote device (PR) and at least one health care professional (HCP) remote device (CP), wherein the RMS comprises a communication module configured to communicate with the at least one remote device, wherein the RMS comprises a central data repository and at least one processor, wherein the central data repository is configured to store data collected by the at least one medical device and the at least one remote device, wherein the RMS further comprises an artificial intelligence (AI) module integrated within the at least one processor and/or cooperating with the at least one processor, wherein the AI module is configured to process data stored in the central data repository and/or to control data communication using the communication module and/or to provide services of a search engine.

At least the object is further solved by a system comprising above defined RMS, at least one medical device and at least one remote device of the group comprising at least one patient remote device (PR) and at least one health care professional (HCP) remote device (CP).

As indicated above the inventive system comprises at least the following components:
At least one individual-patient medical device, for example an IMD, also known as “therapy device”;
A Remote Monitoring Server (or RMS), for example a “Neuro Service Center” (NSC) in one embodiment. The RMS is, for example, a hardware and/or cloud-based service centre that comprises a central data repository of data collected by the at least one CP, the at least one PR and/or the at least one medical device, wherein the hardware may comprise at least one computing device or a network of a plurality computing devices, wherein the RMS further comprises the computing functionality described herein; the RMS is a functional unit that performs substantial computations, including numerous arithmetic operations and logic operations without human intervention and may comprise, such as, for example, a desktop computer, a server computer, clusters/warehouse scale computer or embedded system; the RMS further comprises a communication module configured to communicate with the at least one remote device;
At least one remote device of the group comprising a patient remote device (PR) and an HCP remote device, wherein the PR serves as a transceiver to route communication between the medical device and the RMS, and to provide to the patient some aspect of local control of the therapy process (for example, it gives the patient the ability to change the active therapy program of a medical device, for example a medical device for SCS, control its stimulation amplitude, turn stimulation on/off, and view battery status, it may also serve as an external programmer for the medical device), wherein the at least one HCP remote device/programmer (CP) used by HCPs or field technical representatives to program the medical device remotely. The CP may be physical (i.e., dedicated device) or virtual (e.g., via web-based interface to the system, secure connected data reporters).

The above server and system have the advantage that they improve clinic workflows, quality and profitability. They allow typical clinic employees to access, organize, analyze and report on key patient and practice metrics without the support of dedicated IT personnel. This is expected to result in improved quality of care for the patients and efficiency in the clinic workflow leading to an increased care time budget of HCP. Further, technical resources are used in a more efficient way as the data flow and processing is streamlined.

The system may comprise an integrated network of the at least one medical device, for example an implanted medical device (IMD), the at least one patient remote device, e.g., a transceiver and/or therapy control device, with a patient user interface (UI), the RMS forming a service centre comprising a central processing (and optionally admin) unit, and the at least one CP as an HCP device (e.g., a computer, a tablet or a notebook, also called programmer) with a HCP user interface (CUI) for remotely programming the medical device. The mentioned system components may communicate via public communication networks. Remote programming realizes the use of a CP with a live connection to the RMS and the PR and the medical device.

The present invention may be used in multiple areas of medical device application, e.g., spinal cord stimulation (SCS), cardiac rhythm management (CRM), and many other medical fields that utilize an implanted medical device e.g., deep brain stimulation (DBS), occipital nerve stimulation (ONS), trigeminal nerve stimulation (TNS), vagal nerve stimulation (VNS), phrenic nerve stimulation, gastric electrical stimulation, and sacral nerve stimulation (SNS).

The AI module of the RMS is integrated in the at least one processor and/or cooperates with the at least one processor. In the latter case, the AI module may comprise at least one separate processor, for example a cluster of processors, realizing the AI functionality, wherein the at least one processor of the AI module is electrically connected and communicates signals to/from the at least one processor of the RMS as well as the central data repository. The AI module is configured to process data stored in the central data repository and/or to control data communication using the communication module and/or to provide services of a search engine, wherein the communication may be one- or bidirectional. The AI module may comprise, for example, a “Random Forest” algorithm, where the input is the current data received from the IMD and (0+N) (where N is a natural number; N=0, 1, 2, . . . ) additional data reporters, preset filters for triggering specific actions in response to the data, and historical data stored for that patient and IMD. Output of the algorithm would be the specific actions or reports.

In one embodiment, processing of data provided by the AI module comprises at least one operation of the operations comprising indexing of data, comparison of data, creating an envelope for received data, display of data, and managing data reports.

For example, the AI module provides algorithms and analytics tools to “normalize” data received from medical devices of different models and/or device manufacturers, to allow comparison of behavior and performances between different device models or families. Output of this analysis may drive a presentation layer featuring an intuitive user interface and reports capability to optimize clinical workflow around this analysis.

Indexing of data of the central data repository by the AI module comprises the creation of a database index which is a data structure that improves the speed of data retrieval operations. The indexes are associated with the data of the central data repository, for example a bitmap index, a dense index, a sparse index, a reverse index, a primary index or a secondary index. Indices can be implemented using a variety of data structures. Popular indices include balanced trees, B+ trees and hashes.

In one embodiment, managing of data reports provided by the AI module comprises creating presentation objects, arranging presentation objects in a data report template, creating and storing a data report template, executing of a presentation object or a data report template thereby creating an individual data report and/or exporting data report templates for use in a remote device, e.g., for the HCP of one clinic or a plurality of clinics. This can be denoted, e.g., as “Build Your Own Reports”. For example, the AI includes a “presentation wizard” to provide a range of presentation options for each available data type, creating a presentation object for use in a larger report. Such presentation object is unique to the data being displayed and the preferences of the HCP user. Further, the AI module may comprise a “report wizard” to organize presentation objects into a custom report suitable for the user's needs, for example using drag-and-drop techniques. In a presentation layer the user (for example, an HCP) may view the completed report template. Further, the AI module may comprise “print” and/or “export” wizards to allow for use of the reports outside of the system presentation layer. For example, the report may be used in a remote device, such as CP, PR or external linked data consumer. In the central data repository, a library of standard report templates previously constructed for the clinic may be stored. This allows for creation of a standard default report format for the clinic and for creation of new templates by the individual users. Accordingly, the reports for the data of the central data repository may be individually created and adapted to the user's needs. If one useful report template is created, it can be used any number of times.

The AI module may be capable for interaction with files in their native format (e.g., image files can be used in their native format) because the AI module may create an “envelope” for each data record, such that it can be manipulated and viewed in its native format, for example, within the data reports described above. Accordingly, the AI module is configured to manage most, if not all, common document formats used within the clinic (e.g., image file, XLS, DOC, PDF, etc.).

In one embodiment, controlling of data communication provided by the AI module using the at least one communication module comprises receiving and/or accepting data imports and/or creating and/or sending data exports from/for different data management systems, defining and/or approving at least one external data reporter and/or at least one external data consumer, defining, testing and/or controlling of at least one communication path. This can be denoted as “Build Your Own Connectivity”. The AI module is configured to accept imports or create exports for all generally available data management systems (e.g., electronic health record (EHR), office management software, native central data server), to use a library of common message-type generators and converters. This allows use of a range of common data exchange formats (e.g., XML, HL7, FHIR, etc.). Accordingly, it is not necessary for the user, e.g., an HCP, to choose the required generators and converters, as the underlying AI makes the correct selection for them based upon the overall data routing process that they create. Further, the AI module may provide forms for the user to define at least one external data reporter and/or at least one external data consumer, the data that they communicate and their interrelationships involved in the process. At the end of the defining process such external data reporter and/or external data consumer is approved. For example, an external data reporter may be a chosen group of medical devices and/or an external data consumer may be a data warehouse or a clinical data warehouse. In one embodiment, the user may define connectivity to the data reporter(s) and the data consumer(s) by visually arranging components or controls on a process definition window, specifying attributes and actions for those components. For example, the connectivity processes may be created using drag-and-drop techniques. A tool may be used to place the process controls (e.g., text boxes, buttons, etc.) on the window. These controls have attributes (e.g., a specification that defines a property of an object, element, or file) and event handlers (subroutine that handles inputs received in a program) associated with them. If such connection process is defined, the AI module may provide a “virtual sandbox”. This virtual sandbox allows testing of the created connection to the data reporter(s) and/or the data consumer(s) and its properties before releasing the defined connections for production use. The AI module may further provide tools to define and manage approved data reporters and/or data consumers.

One such connection may be triggering a real-time interrogation of the patient's medical device current data as a remote data reporter in order to provide a remote analysis of current and historical patient data. Another example for a connection to a data reporter may be a remote communication to a particular or to be chosen medical device. Another example for creating a connection to a data reporter and data consumer is the remote programming of the medical device using a CP via the RMS and the PR using a continuous connection for data communication.

In one embodiment of such remote programming of a medical device one or more (e.g., all) steps in the sequence of establishing communication connections between CP and RMS, RMS and PR and PR and medical device may require a “handshake” process that May 1.) ensure cybersecurity and 2.) provide automatic data persistence, communication persistence and communication repetition to ensure that the communication between each component is completed.

In one embodiment, services of a search engine provided by the AI module comprise, beside the indexing of data stored in the central data repository (see above) and, if applicable, of data stored in the at least one approved external data reporter, serving and ranking of search results after receiving a search query. Accordingly, the AI module provides extremely user-friendly search processes that, for example, accept queries as normal text, as well as individual keywords. The user may be, for example, an HCP. Further, the AI module automatically corrects misspelled words, and yields the same results regardless of capitalization. For more customized results, the AI module is configured to use a wide variety of operators found in state-of-the-art search engines (e.g., “OR” to search for webpages containing one of two similar queries, such as marathon OR race, “ ” to force inclusion of a word or a phrase, such as “device recall”. Additionally, the AI module may apply “query expansion” to submitted queries, using techniques to deliver more complete results than the query users actually submitted.

Further, in one embodiment, the at least one remote device may use a submodule which was provided by the AI module. For example, the at least one PR and/or the at least one CP comprises a software subscription module for users (patients and/or HCPs and/or employees of medical device manufacturers) configured to use AI for software subscription management. This AI based software subscription management includes, for example, forms for listing of approved users and their subscription level, automatic generation of user reminders when subscription lapse is pending, automatic installation and release of software modules that users have rights to, automatic update software modules that users have rights to when software updates are released. In one embodiment, a system administrator dashboard may be included to allow for manual management of subscriptions as well as technical support of user issues.

It shall be emphasized that the AI module and the system comprising the RMS with the AI module provides the ability to interact and manage patient data for a complete range of clinic processes in a manner as needed by that individual clinic, i.e., relevant to individual patient historical and longitudinal data, data of their entire patient population, quality control processes for the entire patient, operations control processes for the clinic, business control processes for the clinic, creation of custom reports from entire clinic database, and patient access to limited set of functions.

For communication of the system's components, public communication network(s) (PCN) is (are) used and in each CP an HCP device user interface (CUI), e.g., GUI at the physical CP or web portal at virtual CP and in each medical device a PR user interface (UI). The RMS may further manage deployed at least one PR and at least one CP, which are together called external programmers (EP) in the following.

For data/signal processing each of the CP, RMS, PR, medical device, AI module may comprise at least one processor (processing unit) which is generally regarded as a functional unit of the respective component, that interprets and executes instructions comprising an instruction control unit and an arithmetic and logic unit. The processor may comprise a microprocessor, a controller, a digital signal processor (DSP), an application specific integrated circuit (ASIC), a field-programmable gate array (FPGA), discrete logic circuitry or any combination or plurality thereof. Alternatively, or additionally, the processor may be realized using integrated dedicated hardware logic circuits, in particular in the case of a medical device due to the small size and extreme power limitation.

Each component may further comprise a data memory (which is called central data repository in the case of the RMS) which may include any volatile, non-volatile, magnetic, or electrical media, such as a random access memory (RAM), read-only memory (ROM), non-volatile RAM (NVRAM), electrically-erasable programmable ROM (EEPROM), flash memory, or any other memory device. The data memory may, for example, store buffer data of the respective component as described below.

The medical device may comprise at least one detector detecting at least one bodily parameter of the patient. The detector is connected to the processing unit for processing the determined bodily parameter.

Each component may comprise further units such as a communication module for communication as indicated in this description and a power supply such as a battery. The communication may be at least partly extracorporeally. The communication may be provided wirelessly via the patient's body and/or the air using electromagnetic waves, for example Bluetooth, WLAN, ZigBee, NFC, Wibree or WiMAX in the radio frequency region, or IrDA or free-space optical communication (FSO) in the infrared or optical frequency region or by wire (electrical and/or optical communication). For one- or bidirectional communication the communication module may comprise a receiver, a sender and/or a transceiver. As indicated above, the AI module may provide Build-Your-Own connectivity functionality.

The medical device's, in particular, the implantable medical device's units may be contained within a hermetically sealed housing. This ensures particularly reliable protection of the implantable medical devices and their units against external interference (e.g., moisture).

For remote programming (RP) or interrogation using the CP, the use of single, serial handshakes between each component of the system is significantly reduced. Each component is designed to autonomously maintain a continuous connection to its respective neighbours, via an on-going session that persists for the duration of the RP or interrogation process, wherein the medical device has bidirectional communication link to the PR, the PR has a bidirectional communication link to the RMS and the RMS a bidirectional communication link to the CP, for example:
Medical device and PR maintain, for example a Bluetooth connection while they are in proximity.
PR and RMS maintain, for example a TCP/IP network connection over the internet while mobile networks are available.
CP and RMS maintain, for example a TCP/IP network connection over the internet while mobile networks are available.

Further, feedback loops may exist between the components to maintain a closed loop of one component with the component adjacent to it in the communication pathway, to proactively assess the status of the communication process and to automatically and immediately process information when received. Each link between one component to the next transmits new data to the next hop in chain as soon as new data is available, thereby reducing transmission latency to (nearly) zero. Additionally, in case of connectivity loss, any system component sends new buffered data as soon as connectivity to the neighbour is restored. The system, i.e., each of the at least one CP, the RMS and at least one PR comprises a data buffer of sufficient depth to sustain occasional loss of connectivity, and to allow the transmission of large datasets.

Each link may release local data storage as soon as the neighbour has received the data payload. The process is based upon standard, off-the-shelf communication protocols (e.g., TCP/IP) but the data transmission is controlled by methodologies proprietary to BIOTRONIK and specific to the device application. For example, proprietary data transmission control comprises:
sending alert notifications to request a data transmission, e.g., from the RMS to the PR;
routing data to the right medical device, whereby topology information (connection between PR and medical device) can be used; and
choosing appropriate signature and encryption keys for the receiver of a data transmission.

Component's specifications used will be specific and optimized for each medical device application. For example, the medical device may adjust its data sampling rate based upon the communication bandwidth available to it (communication with PR), the PR may buffer its data transmission to adjust for bottlenecks downstream of it, etc. The concept is similar to how a video feed over the internet is buffered. In typical modern video transmission, TCP and HTTP are ubiquitous, and most video is accessed over the Web. A video server built on top of TCP/HTTP uses commodity HTTP servers and requires no special hardware or software pieces. In the described process, the components are specific to the particular task, and communication is done via a secured network. The system is designed such that no user adjustment is required during the communication process. Each component in the system may automatically optimize its behaviour within a proscribed range to match the current communication capability.

For providing remote programming, in one embodiment, the HCP may trigger the communication session at the CP using CUI via a session request that is sent to the RMS. In one embodiment, the start of the session provided by the HCP may be triggered by the medical device, RP or the patient. For example, the observation of a pathological situation by the patient, the medical device or the RP may initiate a signal comprising the respective information which is then sent by the respective component to the RMS and the HCP. Based on this information, the HCP may trigger the communication session at the CP as described above. Then, the RMS may automatically initiate a session with the PR. Handshake with the PR may be completed and communication between the two components persists. Afterwards, the PR may automatically initiate a session with the IMD. Handshake with the medical device may be completed and communication between the two components persists. The medical device may then send an acknowledgement response to the HCP via the PR-to-RMS-to-CP communication chain. In this embodiment, the session is established, with all components now connected in real-time. Now, the remote programming may be provided to the medical device and/or data may be interrogated from the medical device using the established and continuous communication chain. Upon completion of the use case, the session initiator may indicate that the session is complete and signals the other components to release their handshakes. Throughout this process, only one handshake is required between adjacent components, each communication channel stays open and steady until a closing message is sent, which closes all connections between the devices. In another embodiment the system may work with an analogous method in reverse, with the medical device initiating a session request.

With each system component passing on the data pay load from the device upstream from it as fast as it is received, and refreshing its memory buffer continuously to enable receipt of new data, the information as viewed at the CUI is that of a continuous stream, rather than individual packets of information, and is received with very low latency (i.e., similar to a phone call). Compared to the analogy of “a picture taken and sent via text message” for state-of-the-art IMD systems of sporadically connected sub systems, this present invention provides a “video conference” where data is streamed between the medical device and the CP (and thereby via the CUI the HCP) for the duration of the session.

The RMS may be hosted by a service provider, e.g., a company external from a clinic. The RMS may further comprise at least one of the following services:
Repository of data collected by EP and medical devices;
Repository of data collected by external data reporters;
Repository of keys and certificates for communication of the elements of the system and the HCP or patient via UI or CUI with the system
Facilitates remote programming by routing data to/from their intended destinations;
Communication with an external push notification service;
CP fleet management (e.g., via Airwatch and processes to manage CPs);
Medical device instance authentication service;
HCP, technician and patient authentication service; and
Database analytic tools.

As indicated above, the RMS has a repository of data collected by EPs and medical devices. The latter may comprise home monitoring (HM), which originates from the medical device and is routed to the RMS via the PR. In one embodiment the HM data may be encrypted by the medical device and may only be decrypted by the RMS. In this embodiment, the PR does not have any key to decrypt the data. RMS's “certificate and key management system” (CKMS) may be used to decrypt the data.

Further, after the above mentioned communication connections are established from the CP to the chosen medical device as indicated above, the RMS is used as the conduit to route RP commands from the CP to the medical device and/or return responses and status updates from the medical device to the CP.

A hardware and/or cloud server (e.g., Couchbase® server) may be used to store the data in the RMS. The RMS notifies EPs to pull data from the server via push notifications. RMS sends a push notification request to an external notification service (i.e., Microsoft Azure, Google Firebase Cloud Messaging), which in turn is sent to a specific EP.

A subset of data may be copied from the RMS to a data warehouse as a data consumer to support internal business functions such as technical forensics, quality monitoring, and clinical studies.

CKMS may be used to generate and store secrets for every manufactured medical device. Secrets, e.g., encryption keys and authentication credentials, are generated during manufacturing and stored into the medical device. Generated secrets are used to cipher data when:
Remote programming data is sent from the RMS to the medical device;
HM data are sent from the medical device to the RMS.
RMS uses an authentication management and access service, e.g., Keycloak, for PR authentication. During provisioning, a PR creates a username and password on the authentication management and access service. Subsequently, PRs submit their credentials to the service for an access token, for example, based on JSON Web Token (JWT). The token is then presented to RMS Sync Gateway to gain access. This follows the OAuth 2.0 authorization code grant model. OAuth is an open standard for access delegation, for example, used as a way for Internet users to grant websites or applications access to their information on other websites but without giving them the passwords.

The RMS may provide CP user management and authentication using a Microsoft Azure Active Directory (AAD) which may be integrated in the RMS or may be realized as an external unit connected with the RMS. In the latter case, the RMS interacts with AAD for HCP and patient management operations.

In one embodiment, CPs may follow the OAuth 2.0 authorization code grant model to gain access to the RMS, where AAD acts as the authorization and token endpoints. Once authenticated, the CP is given an access token, which in turn passes to RMS Sync Gateway to gain access.

The at least one medical device may send/receive data to/from the RMS, via the PR, such that the PR may not decipher the data. Accordingly, the medical device may act as a data consumer or a data reporter. Each medical device has unique keys assigned to it at the time of manufacturing.

EPs may communicate to the outside world via either Wi-Fi or cellular network. This introduces a large attack surface that may impact the EP-to-medical device programmability. However, CPs may be protected by the following:
Malware protection and firewall (e.g., Windows Defender malware and firewall).
CP users are granted user rights and thus not able to install software.
Internet resources are whitelisted to resources that are needed for its functionality (e.g., NSC, Microsoft, MDM provider, etc.)
Access to other internet resources, such as Microsoft Store, is inhibited or limited to privately published apps.

PRs may be protected by the following:
No internet browsers installed.
Messaging and phone apps disabled.
Access to other internet resources, such as Google Play Store, is inhibited or limited to privately published apps.

In one embodiment, Couchbase® may be used as the database engine used to store and share data in the RMS. EPs may use Couchbase Mobile, and the RMS may use Couchbase® Sync Gateway and Couchbase® Server. This setup allows for data replication between the mobile client (EPs) and the RMS.

On the PR information may be stored, e.g., in the Couchbase® instance, pertaining to the medical device, which is associated with such aspects as therapy program settings, battery state of charge (SoC), connection state, etc. This data, in turn, is replicated at the RMS.

In one embodiment, when CPs command a change(s) to a target medical device, i.e., a remote programming, the changes are packaged, placed into the local database instance (e.g., Couchbase® instance), and pushed to the RMS. Once received by the RMS, the package is encrypted when authenticated by CKMS using the target medical device's pre-shared secret keys. Because the medical device's shared secret key is only known by CKMS and the medical device, the PR is not able to decipher the package's contents. The PR receives the remote programming package after it has finished synchronizing with the RMS Server.

In one embodiment, data transmission between EP and RMS is encrypted, as well as the BLE link between the EP and medical device. For example, a 2-factor authentication is used when the CP user (e.g., the HCP) selects a chosen patient's medical device to remotely program. In one embodiment, the RMS is configured to store and/or manage the authentication and/or encryption keys and/or certificates for the at least one CP, at least one PR, at least one medical device as well as for the at least one user of the system, for example, patient, HCP and/or technician. For that, the RMS may integrally comprise hard- and/or software solutions for that or the RMS may be connected to respective hard- and/or software solutions. For example, Microsoft Azure may be responsible for managing this on the backend, and Microsoft APIs may be responsible for managing user input on the CP side (i.e., login mechanism, communication with Azure, handling of 2-factor input, etc.).

In one embodiment, once the CP user has been authenticated, an authentication service of the system returns one unique signed token, e.g., the above mentioned JWT token, to the CP (referred to as the remote programming session token). The CP provides this token to the RMS during every remote programming transaction during the remote programming session. The one unique token is valid for the duration of the current remote programming session; subsequent remote programming sessions require a new unique token, which means the HCP having to go through the authentication sequence again. Using such tokens allows decentralizing authentication and authorization.

In one embodiment, the at least one PR may be not configured to continuously poll the RMS for changes, as that would result in unnecessary overhead on both the PR and RMS (i.e., data usage from the PR's perspective and request processing from the RMS's perspective). Instead, the RMS may interface with an external push notification system, such as Google's Firebase Cloud Messaging system, to send a notification to the target PR corresponding to the chosen medical device. Conceptually, for example, prior or at the beginning of establishing a communication connection or at the beginning of the remote programming session the target PR receives a push notification from the RMS. This notification serves as a trigger to the PR to poll the RMS server at a faster rate. In one embodiment, the notification does not contain detailed information, such as any identifying or secret data.

In one embodiment, remote programming session begins with the CP user (e.g., the HCP) logging into their CP. Regardless of a session type, all CP users must log in. After logging in, the CP user will be able to select the option to start a remote programming session, which will present them with a list of patients they may remotely program.

From a hierarchal standpoint, patients may be organized by their clinic. CP users may be associated with one or more clinics. Therefore, CP users may only remotely program patients' medical devices for clinics that they are associated with.

In one embodiment, 2-factor authentication is required to be provided when the CP user (e.g., the HCP) selects a patient to remotely program. For example, Microsoft Azure may be responsible for managing this on the backend, and Microsoft APIs may be responsible for managing user input on the CP side (i.e., login mechanism, communication with Azure, handling of 2-factor input, etc.). The CP is responsible for sending a request to authentication service to gain access to the remote programming resource. Once the user has been authenticated, the authentication service returns a signed token, e.g., a JWT token to the CP (referred to as the remote programming session token). The CP provides this token to the RMS during every remote programming transaction during the remote programming session. The token is valid for the duration of the current remote programming session.

Once the code has been authenticated, the CP may pull the latest copy of the target medical device's data from the RMS, and the user will be able to proceed with a remote programming session.

The following data may be used in the AI module for data processing and storing in the central data repository of RMS as well as transmitted during a remote programming session (i.e., “signals”). Please note that these data will differ dependent upon the embodiment. The following data may refer partly to an SCS system which is described here as a typical embodiment.
A) Medical device data
   Medical device data (serial number, medical device type)
   Therapy data (e.g., therapeutic data (packed parameter values, packed data elements, and interface parameters), lead impedance, offset measurements, therapy program selection, stimulation on/off, enable/disable MRI mode, battery state of charge (SoC), status changes, lead info/configuration (“blob” and interface data), medical device alerts, IMD EC App Status, stimulation usage data (used for statistics)
   Personal data (patient name, physician name, hospital, implantation date of the medical device, indication, lead position, lead type and extension information, implanted system component traceable IDs
   Notes
   Patient Identifier (unique ID, serial number of medical device)
b) Technical data
   Technical data/logs (reset, battery, etc.)
   System-based events
   Battery history log
   MRI mode
   Magnet history
   medical device reset log
   EP application log
   application logs
   Error logs
   Audit logs
c) Connectivity device data for communication protocols (the system uses the standard data overhead required for the following communication protocols)
   Bluetooth® Low Energy (BLE) (the medical device radio's transceiver may support
   BLE 5.0, though the product uses BLE 4.2)
   4G
   Wi-Fi
   TCP/IP

The RMS is configured transmits at least part of these data from the RP while the RMS transfers data with the CP.

In one embodiment, for remote programming of the chosen medical device the CP is configured to produce a single program containing the updates and/or changes of the medical device's parameters and to transmit the single program to the medical device via the RMS and the corresponding PR. This is explained in more detail below. Further, in one embodiment the RMS is configured to encrypt the single program received from the CP, wherein the corresponding PR is configured to transmit the encrypted single program to the chosen medical device. Accordingly, the PR cannot decrypt the single program. Decryption is provided by the medical device as explained in detail below.

In one embodiment, the RMS is configured such that its central data repository comprises data having different security level, wherein one example of data with a lower security level are pseudonymized data. Data with a higher security level are personal or private data such as names, addresses, user ID etc. These data need higher protection and can only be accessed by persons having a higher security level.

In one embodiment, in order to provide data of the RMS for further analysis or for assessment of efficacy of the therapy of the at least one medical device, the RMS is configured to be connected with a data warehouse and/or a clinical data warehouse as data consumer, wherein the data warehouse and/or the clinical data warehouse are configured to import at least a part of the data stored in the central data repository. The warehouses and their operation are further explained below.

Further, at least the above object is solved by the method (i.e., a computer-implemented method) for operating a remote monitoring server (RMS) configured to be used in a system comprising at least one medical device and at least one remote device of the group comprising at least one patient remote device (PR) and at least one health care professional (HCP) remote device (CP), wherein the RMS comprises a communication module configured to communicate with the at least one remote device, wherein the RMS comprises a central data repository and at least one processor, wherein the central data repository stores data collected by the at least one medical device and the at least one remote device, wherein the RMS further comprises an artificial intelligence (AI) module which processes data stored in the central data repository and/or controls data communication using the communication module and/or provides services of a search engine.

In one embodiment, processing of data provided by the AI module comprises at least one operation of the operations comprising indexing of data, comparison of data, creating an envelope for received data, display of data, and managing data reports.

In one embodiment, controlling of data communication provided by the AI module using the at least one communication module comprises receiving and/or accepting data imports and/or creating and/or sending data exports from/for different data management systems, defining and/or approving at least one external data reporter and/or at least one external data consumer, defining, testing and/or controlling of at least one communication path.

In one embodiment, managing of data reports provided by the AI module comprises creating presentation objects, arranging presentation objects in a data report template, creating and storing a data report template, executing of a presentation object or a data report template thereby creating an individual data report and/or exporting data report templates for use in a remote device.

In one embodiment, services of a search engine provided by the AI module comprises, beside the indexing of data stored in the central data repository and, if applicable, of data stored in the at least one approved external data reporter, serving and ranking of search results after receiving a search query.

The above embodiments of the operation method have the same advantages as the above system. Embodiments of the system indicated above may be realized in the operation method analogously. It is referred to the above explanation of the system in this regard.

The above method is, for example, realized as a computer program which comprises instructions which, when executed, cause the processing units (processors) of the components of the system to perform the steps of the above method which is a combination of above and below specified computer instructions and data definitions that enable computer hardware to perform computational or control functions or which is a syntactic unit that conforms to the rules of a particular programming language and that is composed of declarations and statements or instructions needed for a above and below specified function, task, or problem solution.

Furthermore, a computer program product is disclosed comprising instructions which, when executed by the processing units (processors), cause the processing units to perform the steps of the above defined method. Accordingly, a computer readable data carrier storing such computer program product is disclosed.

Additional features, aspects, objects, advantages, and possible applications of the present disclosure will become apparent from a study of the exemplary embodiments and examples described below, in combination with the Figures and the appended claims.

### BRIEF DESCRIPTION OF THE DRAWINGS

The present invention will now be described in further detail with reference to the accompanying schematic drawings, wherein

FIG. 1 shows one embodiment of the inventive system and the steps establishing the communication between the components of the system;

FIG. 2 depicts another sketch of the inventive system;

FIG. 3 shows a remote programming overall workflow;

FIG. 4 depicts an example of 2-factor authentication using push notification based approval;

FIG. 5 depicts a flow diagram of initiating an RP session (CP to PR);

FIG. 6 shows a sequence diagram of initiating an RP session (CP to PR);

FIG. 7 shows a flow diagram of RP session, namely PR to CP response;

FIG. 8 depicts the RP session communication CP to PR to medical device;

FIG. 9 shows a sequence diagram of RP with transmission acceptance;

FIG. 10 shows a sequence diagram of RP with transmission rejection;

FIG. 11 shows a sequence diagram of RP transmission with connection loss;

FIG. 12 shows an overview of another embodiment of an inventive system;

FIG. 13 depicts the embodiment of FIG. 12 in more detail regarding the system architecture;

FIG. 14 shows a system data workflow and access diagram of another embodiment of the inventive system,

FIG. 15 shows an example of a session summary report;

FIG. 16 shows an example of a programming details report;

FIG. 17 shows an example of another session summary report (detail);

FIG. 18 shows an example of another session summary report; and

FIG. 19 shows an example of a CP screen during RP (program updated awaiting transmission).

### DETAILED DESCRIPTION

In the following embodiments of the inventive system will be explained in detail. The embodiments are explained with regard to medical devices in the form of spinal cord stimulation (SCS) devices and a remote monitoring server (RMS) in the form of a Neuro Service Center (NRS) and with regard to remote programming which may include data interrogation.

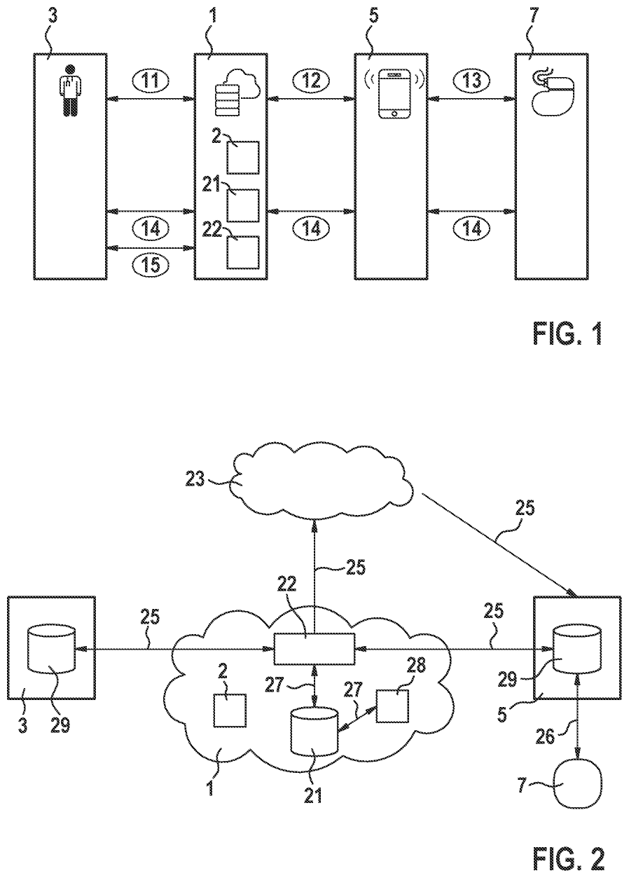

FIG. 1 shows the components of the system, namely the RMS 1, which is, for example, the NRS, at least one HCP remote device (CP) 3, at least one patient remote device (PR) 5, and at least one medical device, for example an SCS device, 7. For further information and for the properties with regard to the system components 1, 3, 5, 7 it is referred to above explanation in the general description. The RMS 1 comprises an AI module 2, a communication module 22 and a central data repository 21, wherein the AI module 2, the communication module 22 and the central data repository 21 are connected to each other and provide data exchange (communication). The AI module 2 shown in FIGS. 1 and 2 generally comprises the features described in the general description of the present invention above.

Using the AI module 2 a data import/export/communication process may be defined as, for example, the remote programming method as explained in the following. Each communication step of the remote programming as described in the following may be defined using the AI module 2 as well as the data imports and exports as indicated below. Data reporter may be, for example, the CP 3 and the medical device 7, wherein data consumer may be the medical device 7 and the RP 5. Further, the AI module of the RMS may be used to define and approve data report templates showing the success of the remote programming of the medical device 7 and the parameters that were changed with the remote programming steps.

For providing the different functionalities of the AI module 2, the AI module 2 comprises a “Remote Monitoring Server Analytics” clinical practice management tool that provides a number of “dashboards” that assist the HCP or the clinic in managing patients and their practice, for example:
A “Clinical dashboard” for interacting with the data and management of individual patients;
A “Connectivity dashboard” for management of data exports from and data imports to the clinic database;
An “Operations dashboard” for interacting with the data and management of the entire patient population of the clinic, to identify trends and opportunities for improved care (e.g., helps you identify device patient populations that are not complying with clinical guidelines, or are using their devices in a sub-optimal manner);
A “Productivity dashboard” for tracking key business metrics for the clinic (e.g., trends patient volumes over time, implantation of devices and patient encounters, patients lost-to-follow-up, reimbursement capture, etc);
A “Quality dashboard” for tracking key quality metrics for the clinic (e.g., trends complications and contributing factors over time by implantable device type, model, and provider; tracks recalls); and/or
A “Kiosk dashboard” for patient interaction (e.g, bill paying) a dashboard for clinic support provided by the medical device company representatives.

During programming (i.e., creating the program by the HCP using the CP) the HCP may use the search engine functionality of the RMS 1/AI module 2 described in the general description above in order to get an overview of the present patient's situation.

Additionally, the data received by the RMS 1 (see examples below) and then stored in the central data repository 21 may be processed and indexed by the AI module 2 in order to provide these data for the search engine functionality or reports as explained in the general description above.

An overview of the remote programming (RP) method defined by the AI module 2 may be derived from FIG. 1 comprising the following steps. At first, the HCP triggers the communication session at his/her UI (CUI) at the CP 3 by a session request 11 which is sent to the RMS 1. Then, the RMS initiates a bidirectional communication connection 12 with the PR 5 based on a handshake with the PR 5. In the next step, the PR 5 initiates a communication connection 13 with the medical device 7 based on a handshake process, as well. Then, the medical device 7 sends a response to the CP 3 via the PR 5 to RMS 1 to CP 3 communication chain which is denoted in FIG. 1 with reference number 14. Now a continuous bidirectional communication connection is established between the CP 3 and the medical device 7 with all components now connected in real-time. Upon completion of the use case, the session initiator (HCP) indicates at CP 3 that the session is complete and signals the other components to release their handshakes (step 15). Throughout this process, only one handshake is required between adjacent components, each communication channel stays open and steady until a closing message is sent which closes all connections between the components.

In one embodiment the following protocols may be used:
Medical device 7 to PR 5: Bluetooth Low Energy (BLE)
PR 5 to public communication network(s) (PCN), e.g., 4G LTE, WiFi 802.11
PR 5 to RMS 1: TCP/IP
RMS 1 to public network(s): VPN
RMS 1 to CP 3: TCP/IP

Accordingly, the system components of this embodiment and other embodiments may comprise interface modules to communicate via mobile wireless networks (e.g., SMS connection, GPRS data connection, UMTS data connection, LTE data connection), virtual private network(s) or dedicated line(s), internet and local networks via Ethernet or WLAN, electronic patient/case files (electronic medical records) via HL 7 v2 or v3 or similar. Further, suitable communication standards e.g., TLS+TCP/IP, SSL+TCP/IP or ebXML may be used.

FIG. 2 contains a different visualization of the whole inventive system. The RMS 1 comprises a Couchbase server 21 as central data repository to store data. Additionally, RMS 1 comprises a Couchbase Sync Gateway 22 for synchronization and communication with CP 3 and PR 5. The system further comprises External Push Notification Services 23 (e.g., Microsoft Azure, Google Firebase Cloud Messaging) in order to notify the CP 3 or the PR 5 to pull data from the RMS 1 via push notifications. For that, the RMS 1 sends a push notification request to the external push notification services 23. In FIG. 2 the arrows 25 represent Internet connections, the arrow 26 a BLE connection and the arrows 27 local network connections (e.g., LAN connections). The RMS 1 further comprises a certificate and key management system (CKMS) 28 which is used to decrypt the data received from the at least one medical device 7. In one embodiment the External Push Notification Services 23 may be integrated in the RMS 1. Further, in order to communicate with the Couchbase server 21 of the RMS 1 the CP 3 and the PR 5 each comprises a Couchbase mobile application 29.

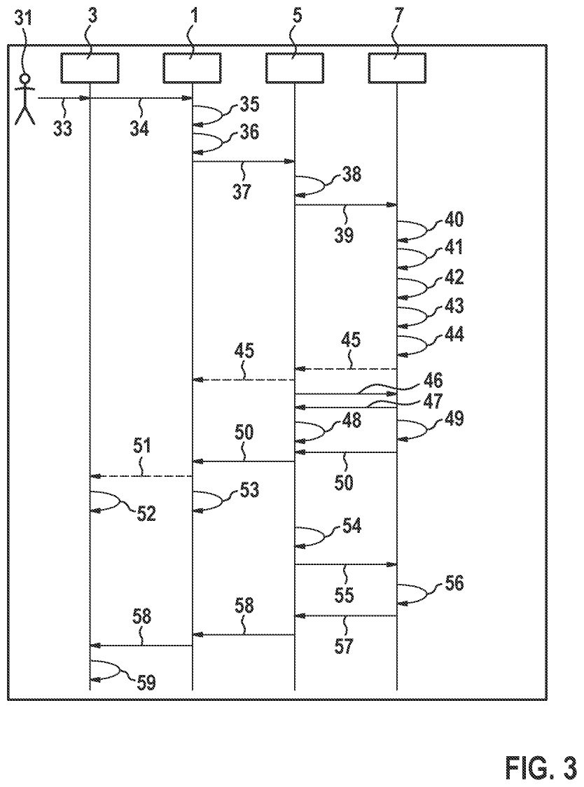

FIG. 3 shows an overall workflow for remote programming which is explained in further detail in the following. The workflow for remote programming works provided the continuous communication connection is previously established between the CP 3 and the medical device 7 after selection of a specific patient/medical device 7 for remote programming. The sequence of events starts with the CP user (e.g., an HCP) commands the transmission of a therapy program using remote programming. As indicated below, the therapy program contains all changes/updates of the parameter of the medical device 7 which are to be programmed. The method describes the process uplink and downlink.

At first, after authentication of the HCP 31 at his/her CP 3 the HCP 31 configures a new therapy program for the patient to be used at its medical device 7. Then, after pressing the transmit button (step 33) the therapy program is transmitted to the RMS 1 (step 34). At the RMS the access token is verified (step 35) and a remote package containing the information of the therapy program is ciphered (step 36). This remote package is then transmitted (automatically, i.e., without further HCP or patient interference) to the PR 5 (step 37). In the next step, the patient is prompted about the fact that there is a therapy program arrived at his/her PR 5 (step 38). In the next step 39 the patient accepts the therapy program and the ciphered remote package is automatically sent to the medical device 7. The remote package is not deciphered at the PR 5 but at the medical device in step 40. In step 41 the therapy program is saved to a buffer of the medical device 7. Step 42 comprises ruling a check to the therapy program in medical device 7 and step 43 and installation and activation of the therapy program. Additionally, in step of 44 the medical device 7 generates and ciphers a remote acknowledgment signal which is to be sent to the RMS 1.

Then, using the continuous communication connection to the RMS 1, the encrypted acknowledgment signal is returned to the RMS 1 via the PR 5 (see steps 45).

Additionally, the PR 5 may automatically interrogate the medical device 7 for new data. This is facilitated in step 46, wherein this step contains an interrogation request to the medical device 7. This interrogation request may be sent via the continuous communication connection between the PR 5 and the medical device 7. In response to this request in step 47 the medical device 7 provides the PR 5 with its interrogation data. Then, in step 48 the PR 5 updates its graphical UI. At the same time the medical device 7 generates and ciphers HM PGMR frame in step 49. The PGMR frame contains detailed parameter values programmed in the medical device. In step 50 the PGMR frame is returned to the PR 5 and routed to the RMS 1. In the next step 51 the latest therapy entities may be pulled to the CP 3. Then, in step 52 the CUI of the CP 3 is updated and at the same time in step 53 the PGMR frame deciphered, data assembled, PPVs unpacked and PDEs as well as appropriate entities at the RMS 1 updated. This allows to check the success of the remote programming procedure on the CP and to give feedback to the HCP via the CUI.

In the next step 54 further adjustment of the medical device 7 may be provided by the PR 5, for example, in the case of an SCS a global amplitude may be adjusted. A respective signal, for example, to adjust the medical device's program stimulation amplitudes and to save the global amplitude may be sent to the medical device 7 in step 55. The respective action is provided by the medical device in step 56. In step 57 a response is sent from the medical device 7 to the PR 5 returning stimulation amplitudes. The following steps 58 returns the latest status of the PR 5 to the RMS 1 and the CP 3. Finally, in step 59 the CUI the of the CP 3 is updated accordingly.

In the process described with regards to FIG. 3 the steps 34, 37, 51 and 58 may be Couchbase, commands and responses. Further, the steps 39, 45, 50 may be provided as end-to-end encrypted commands and responses.

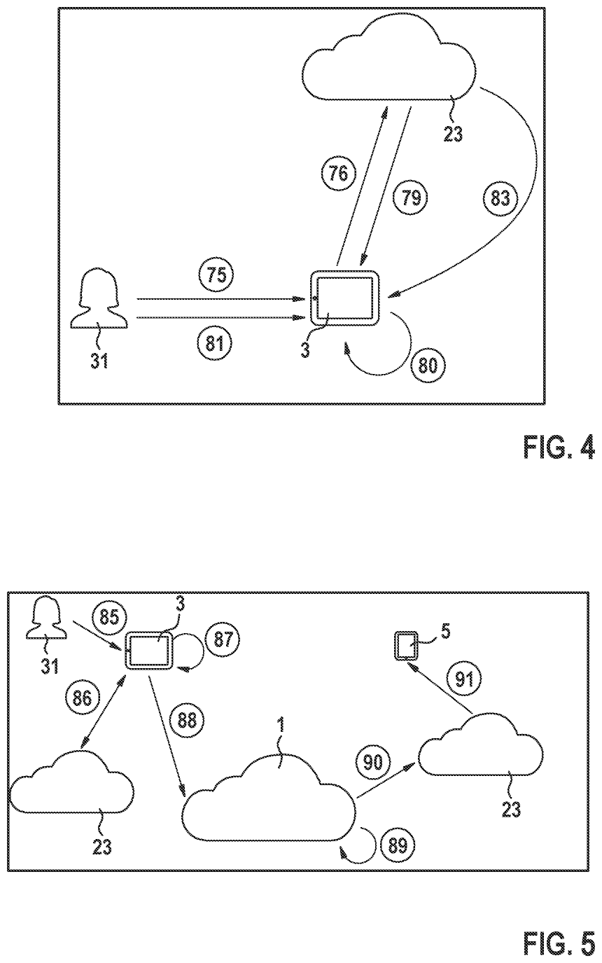

Prior to above programming steps the remote programming process needs to be initiated which is explained in the following with regard to FIG. 4 showing the start of a remote programming (RP) session after the HCP was logging into his/her CP 3.

Patients may be organized by their clinic. HCP, i.e., CP users, may be associated with one or more clinics. Accordingly, it may be defined that a CP user is allowed to remotely program patients medical devices only for such clinics to which they are associated. In one embodiment, a 2-factor authentication is required to be provided if the HCP selects a patient for remote programming. As indicated above, Microsoft Azure may be responsible for managing this on the back end and Microsoft APIs may be responsible for managing user input on the CP side (login mechanism, communication with Azure, handling of second factor input etc.).

The RP session starts with the HCP requesting to start remote programming in step 75 in FIG. 4. Then, in step 76 user credentials are passed for a remote programming token to the external push notification service 23. In step 77 this service sends an authorization request to the HCP's mobile device and in step 78 the HCP 31 accepts the request. Then the external push notification service 23 returns a signed access token to the CP 3 in step 79. The process then continues with remote programming (step 80).

The token (MFA token) provided by the external push notification service 23 to the CP 3 is a signed JWT token which is also referred to as the RP session token or access token. The CP 3 provides this token to the RMS 1 in every RP transaction during the RP session. The token is valid for the duration of the current RP session, subsequent RP sessions require a new token which means having to go through the authentication sequence described above again.

In the following steps, the components of the system execute connectivity steps. For that, the following messages/information are utilized by the system and/or the method:
Follow-up ID (a unique identifier for each follow-up session, used for system processing and tracking)
The above mentioned multifactor authentication token (MFA, i.e., the token of the RP session determined by 2-factor authentication).
RpStartSession message: an entity requesting the start of a remote programming sequence and contains information to specify the target patient. This is used when the HCP selects a patient on the CUI for the remote session. RMS's remote-programming service triggers the associated PR 5 with a push notification as explained above. The PR 5 may start a replication, in case there are a lot of un-replicated data to catch up with.
RpCpRequest message: containing the therapy program parameters
RpPrRequest message: containing encrypted programming commands
RpRmsStatus updates CP 3 with programming progress; captures the status of RMS's processing of the RP request with various timestamps, etc. This message contains, for example, time of RpCpRequest received, time of RpCpRequest signed by CKMS, time of RpPrRequest generated, time of push notification to PR sent, other state and error info
RpCpStatus. The RpRmsStatus and RpPrStatus entities are used to communicate status at each hop. Each entity may contain tasks that each hop is responsible for performing, errors/status values, completion of tasks denoted by a timestamp.

The remote programming method further makes use of a “Time to Live” calculation (TTL) in its control process. As indicated above, TTL is a value in an Internet Protocol (IP) packet that tells a network router whether or not the packet has been in the network too long and should be discarded. This TTL is an absolute time in UTC. TTL is based on PR's 5 time to account for time synch mismatches between CP 3 and PR 5. TTL is calculated using current PR time acquired at session start and elapsed time since RMS data base synchronization and timeout interval. When PR receives RpPrRequest entity, it takes the TTL value and starts a timer. Expiration of TTL is communicated by setting the respective value in the RpPrStatus entity.

With regard to FIG. 5 further steps during initiation of an RP session are explained. This flow diagram shows that the PRs 5 are not configured to continuously poll the RMS 1 for changes as that would result in unnecessary overhead on both the PR 5 and the RMS 1. Instead, the RMS interfaces with the external push notification service 23 to send a notification to the target PR 5. For example, at the beginning of a remote programming session the target PR 5 receives a push notification. This notification serves as a trigger to the PR 5 to poll the RMS 1 (i.e., its Couchbase server 21) at a faster rate. The notification does not contain detailed information such as any identifying or secret data.

For example, the following steps may be executed for remote programming (i.e., remote follow-up). At first, the HCP selects a patient to program in step 85. The HCP navigates to the remote patient management page on the CUI found on the CP 3 and selects a patient from a filterable list of opted-in patients who the HCP is authorized to follow-up with. Then, the 2-factor authentication is provided as indicated with regard to FIG. 4 above (step 86). At the same time, the CP 3 creates an entity requesting the start of an RP session (step 87), i.e., the CP 3 sends a RpStartSession command to the RMS. This means that an uplink is created from the CP 3 to PR 5. The CP 3 then waits for successful authentication, and for the PR 5 to respond with an object of the medical device 7 of the patient via RMS 1. Then, the RpStartSession is synchronized with the RMS 1 (step 88). After that, the RMS 1 identifies the target PR 5 which corresponds to the selected patient (step 89), e.g., using Google firebase. Then, a push notification request is sent to the external push notification service 23 (step 90), which contacts the PR 5 corresponding to the selected patient and the PR 5 receives the push notification and is thereby triggered to replicate with the RMS 1 at a faster rate (step 91)

As a response, which is depicted in FIG. 7, the PR 5 synchronizes with the RMS 1 (step 93) after the current PR time and medical device connection state was placed into a response entity (step 92). For example, the current battery state, therapy data such as stimulation state, and/or MRI mode state, and any medical device errors or other data of the PR database will be sent from the PR 5 to the RMS 1. Similarly, the CP 3 synchronizes with the RMS 1 (step 94), wherein the above-mentioned data are further transmitted to the CP 3. Additionally, the CP 3 quotes data from relevant entities to start the remote programming session (step 94 a). Updates to this information will be delivered on a change in any of these items. If the CP user (e.g., HCP) has not begun editing a program, the PR will also update the CP via RMS 1 with any changes to the active program, program strength, or program start strength. Relevant status information will be logged using the normal logging mechanisms. If the response of the PR 5 is received within a pre-defined time (e.g., 30 seconds) of the CP 3 sending the RpStartSession command, and the response indicates that PR is connected to the medical device and has the expected follow-up ID, the CP enters the standard programming page in remote programming mode. Otherwise, CP displays an appropriate message and stays in the remote patient management page.

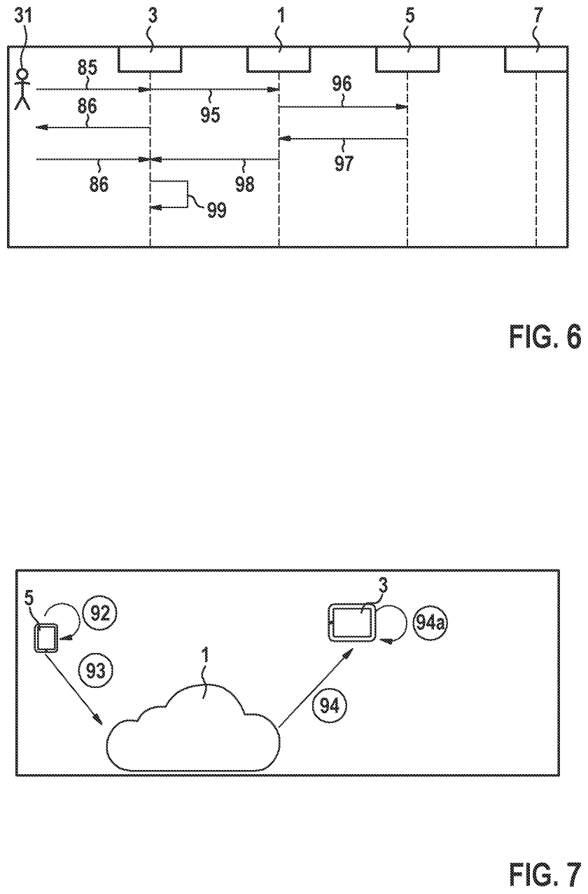

FIG. 6 shows the start of the remote programming session as a sequence diagram which is explained in the following. As indicated above the HCP 31 first selects a patient who needs to receive a program update of his/her medical device 7 (step 85). The 2-factor identification illustrated above is provided by the steps 86. Then, a start session command sent from the CP 3 to the RMS 1 (step 95). In the next step 96 the RMS 1 sends a start session notification to the PR 5. Triggered by this command the PR 5 responds by sending information received from the medical device 7 previously (step 97). This information, for example, may comprise current battery state, stimulation state, MRI mode state, or any medical device errors. This information is further transmitted to the CP 3 in step 98. Further, CUI is updated in step 99 afterwards.

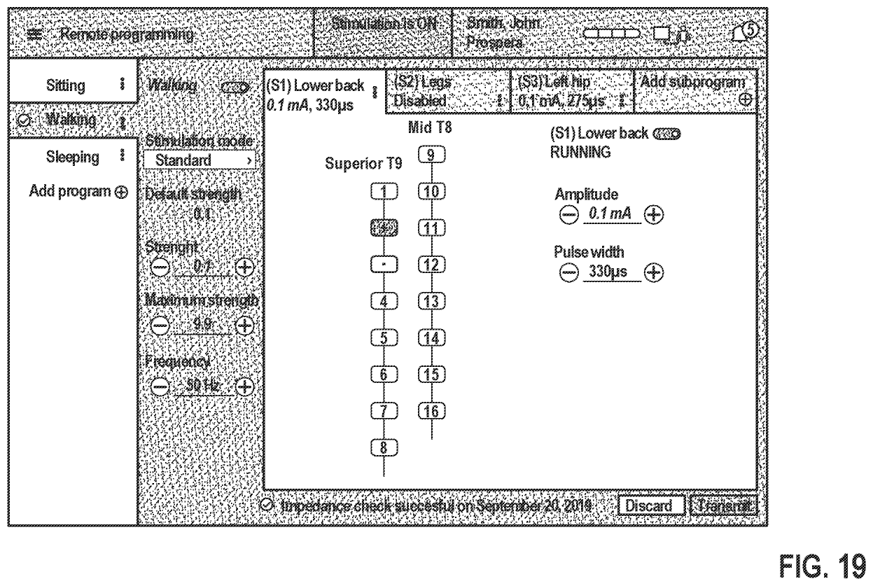

In the remote programming mode CP application stages parameter changes provided by the HCP 31 to a single therapy program using the programming CUI. Remote programming differs from face-to-face in that instead of transmitting changes immediately pending changes will be highlighted to indicate to the HCP 31 the changes that will be made. This can be derived from FIG. 19 which contain an example of a programming screen of a CP 3. Attempting to switch programs prompts the HCP as the CP user to discard his/her changes or stay on the current program. If the HCP has made all desired changes and taps the transmit button a ‘transmission in progress’ overlay may be displayed to prevent navigation or further program edits. This overlay may persist until programming completes will be HCP chooses to end the session.

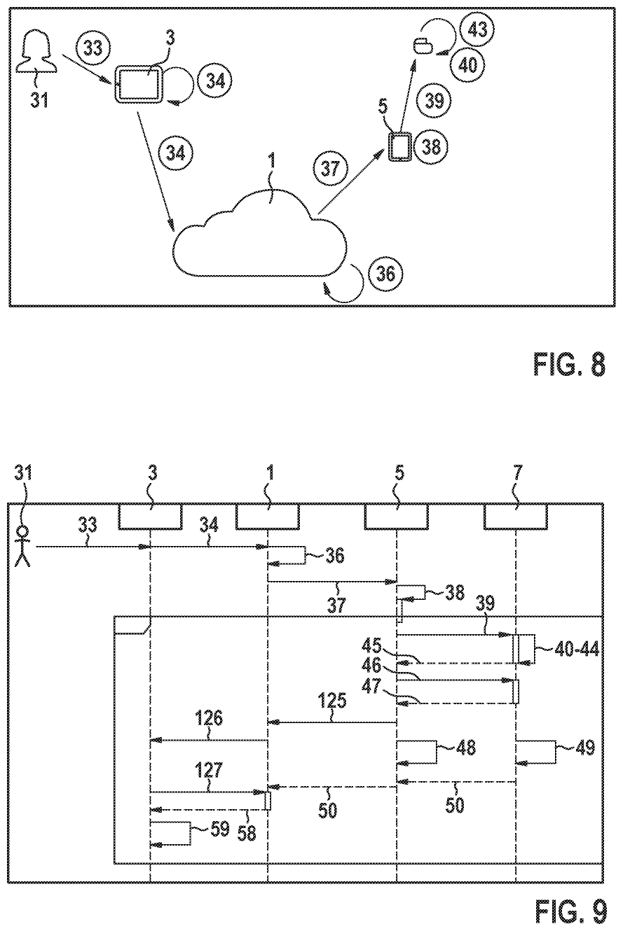

The following steps of remote programming are visualized in FIGS. 8 and 9. Pressing the “Transmit” button starts the remote programming using the established communication chain CP-to-RMS-to-PR-to-medical device. The following steps describe uplink and downlink.

For the safety and comfort of the patient, all programs updated through remote programming will have their program strength set to a pre-defined minimum value. After successful programming, the patient may be required to adjust the medical device parameters, for example the strength and default strength to an appropriate level, using the standard PR 3 UI. If the remote programming session is still in progress, these changes will be reflected in the CP 3, as described above.

After pressing the transmit button (step 33, see FIG. 9), to perform the transmission, the CP 3 creates and sends an RpCpRequest entity, containing the therapy program parameters, to the RMS 1 (step 34). After program encryption (step 36), the RMS 1 then sends an RpPrRequest to the PR 5 containing encrypted programming commands (step 37) and creates an RpRmsStatus to update CP 3 with programming progress. The RMS 1 starts a Push Notification to the PR 5 so that the PR 5 may start a replication to get the RpPrRequest update as indicated above. The PR 5 does not parse this data. Upon receiving this request, if the TTL has not expired, the PR 5 asks the patient to accept or decline the program update (step 38).

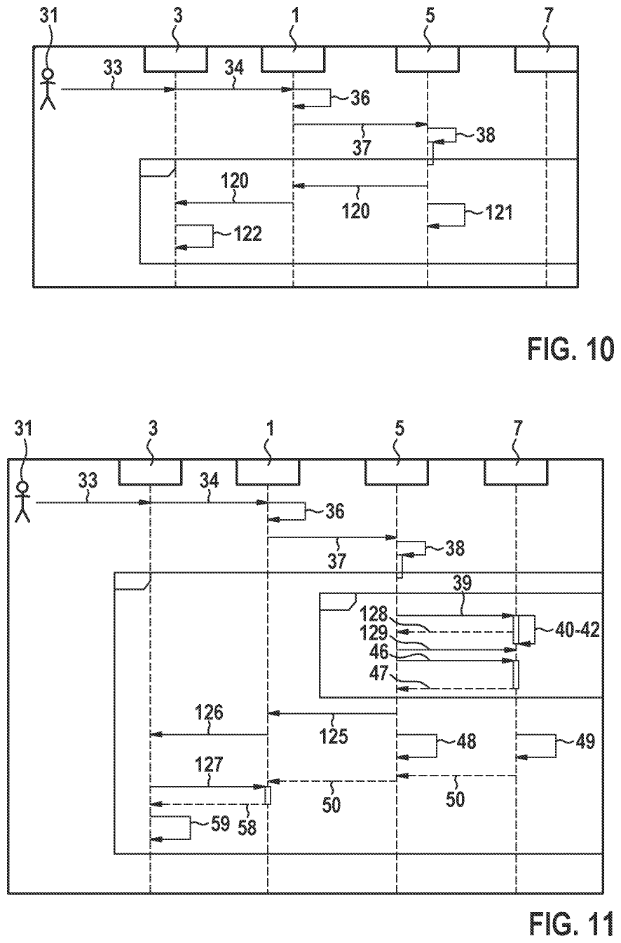

If the user rejects the update, which is shown in FIG. 10, the PR 3 will insert an RpPrStatus entity indicating the user rejected the update and a message is displayed to the PR 5 and CP 3 users via RMS 1 (steps 120). Accordingly, the UI of PR 3 and the CUI of CP 3 are updated (steps 121 and 122).

If the patient accepts the request in step 38 (see FIG. 9), the PR 3 sends the data to the medical device 7 using, for example, Bluetooth (step 39). The further steps 40 to 44 have already been explained with regards to FIG. 3. Upon completion the PR 5 will resynchronize with the medical device 7 (steps 46 and 47) and sends an RpPrStatus to the RMS 1 to indicate the result of the programming operation (step 125). If the follow-up ID or revision has changed, programming is considered to be successful, otherwise programming is determined to have failed. On successful programming, the CP 3 then receives an updated Follow-up entity from the RMS 1 which is loaded to continue the session (step 126). The following steps 58 and 59 have already been explained in connection with FIG. 3. If programming does not succeed, the CP 3 continues the session as it was prior to the “Transmit” button being pressed. The HCP uses the side menu end session option to end the session once all desired edits have been transmitted.

During remote programming, it is possible for the PR 3 to lose connection to the medical device 7 which is depicted in FIG. 11 in step 128. In this case the PR will wait until a connection has been reestablished (see step 129 in FIG. 11) and then resynchronize with the medical device 7. If after the completed resynchronization the follow-up ID or revision has changed, this will indicate successful programming. If the follow-up ID or revision has not changed the PR will retry the programming operation.

If, during remote programming, the CP 3 receives a RP 5 or medical device 7 update, or other entity, which indicates the follow-up ID or revision has changed to an unexpected value, the CP 3 will immediately notify the HCP and end the session. This scenario could occur if multiple CP's attempt to conduct remote programming sessions with the same PR 5 at the same time.

Some information needed for remote programming changes very frequently and thus the system does not/may not rely on the standard home monitoring process to transfer this information. For the purposes of RP 5, it needs to be up-to-date at the beginning of the remote programming session but it also needs to be continually updated after the remote programming session so that the CP 3 may see the patient amplitudes change in real time.

The system further allows combining subjective (e.g., patient diary, patient surveys, HCP notes) and objective data (e.g., current patient status, patient trends and outcomes analysis, longitudinal pain data, activity-based tracking, medication use checking, statistics) into one viewable RMS database via multiple data reporters, for example, CP 3, PR 5, RMS 1 (for example, NSC), Electronic Health Record system (EHR) and to process these data using the AI module 2. Further, the RMS 1 provides a central service unit that serves as a central data repository 21 of all of clinic's patients, as well as manages access rights and controls access to therapy device data according to user's log-in. The system may further comprise a web-based presentation layer, accessible from (1+N) different UI. The system may further provide HCP-accessible reporting which includes controlled access to database, configurable access rights within the database (i.e., to specific patient or groups of patients), accessible via multiple portals (CP, NSC UI, EHR UI, other) using customized reports as described above. The HCP may view both current patient status as a real-time “snapshot” as well as via long-term data trends. The reporting may further comprise configurable report formats to allow customization to clinic or individual user's needs. The system further comprises a searchable patient and medical device database indexed based on AI algorithms.

In the following one example of data organization/management in the RMS 1 is explained in detail also with regard to data security.

In connection with this, it turns out that an important feature of the system is that all data within the system may be organized such in the RMS 1 that they are accessible to employees of an external service provider (technician of the system, external HCP, statistics companies, for example, for patient's studies, companies offering medical devices which may receive further information on the behaviour of their product(s)) via certain tools/interfaces (e.g., a Data Warehouse (DWH), tools for the customer service, etc.), for example, for the purpose of post market surveillance of the used medical devices. The accessible data also include serial numbers, names, dates, etc. Accordingly, access rights are implemented to allow access to exactly that data that each employee needs in order to perform the task at hand. By offering all data via the interfaces of the RMS 1, the necessary access rights and related processes for being compliant with privacy laws may be defined independently of the design, development and maintenance of the RMS.

The concept mainly relies on two principles, namely, to categorize data as close to the origin as possible (i.e., CP, PR, RMS) into different data sets (see “Categories of Stored Data”), restrict access to data as close to the data user as possible (e.g., DWH and Customer Service Tools), based on clearance of use.

The data may be accessed by the sub-systems Data Warehouse (DWH) und Clinical Data Warehouse (CDW) as external data consumers. If patients have given explicit consents for being included in studies, the AI module may define and/or approve a DWH for importing respective data from the RMS. These data may be offered to service provider's internal users based on the user's access rights. The CDW may be defined such that it imports pseudonymized and device identification data from those patients that are part in studies.

RMS may comprise customer support tools for the customer service. These tools may be used for 1st level troubleshooting, customer support and customer administration. There may be an RMS frontend that allows/requires the HCP to directly interact with the RMS 1. In one embodiment external data reporters/consumers will allow for the secure exchange of data between external data reporters/consumers and the RMS.

The data in the RMS, the DWH and the CDW may be stored encrypted. Access rights are in place to ensure restricted access to the stored data. Physical access to the data bases is restricted to administrative staff. The RMS frontend end the RMS customer support tools do not store data themselves but may always request the data live from the RMS 1.

In one embodiment, each access point has a UI designed specifically for the functions performed.

The AI module 2 of RMS 1 may also define data communication of the RMS 1 and the at least one medical device 7 as a closed loop, real-time communication. For example, if the HCP wants to know the patient's data in preparation for follow-up consult or remote assistance call with the patient, the defined communication may allow HCP to trigger a respective request (update data request) from the RMS 1. Further, the medical device 7 is defined as a data reporter with regard to the request. This request may then be transmitted to the PR 5 which in turn relays the request to the medical device 7. The medical device queries current patient and device status and transmits the report to the RMS 1 via the PR 5. The report is received and processed (i.e., the message is converted to form usable by the RMS 1, scanned for alert conditions using internal algorithms and assigned to the correct clinic and patient) by the RMS 1. Then, the HCP this able to review the new report in advance of the in-person meeting. The interface may be used to display data received via regular routine to connect data automatically (daily, monthly, . . . ) or on purpose (e.g., preparation for in-office visit, patient adjustment, . . . ). Further, the RMS 1 is configured to provide a current patient status ‘snapshot’ collected in near real-time showing all quantitative and subjective data store for the patient. In addition, as indicated above, the RMS 1 provides online access to the PR and the medical device to collect real time data. The RMS 1 is further configured to provide the status of the patient's system and statistics collected over time (e.g., for an SCS device implanted lead integrity, present stimulation settings, the diagnostic trends related to pain or system use). In one embodiment, the user may have the ability to configure the display of the combined status such that different data trends are visible or invisible according to the user's preferences. The HCP's web interface of the RMS 1 may further provide an image of the patient, videos or audio files. Further, the interface may allow to query the database, for example for all patients or medical devices under control of the respective clinic.

Additionally, the RMS 1 may provide the feature of an electronic signature ability to allow the HCP and/or patient to electronically provide consent for future participation in remote follow up or scientific studies. Further, the RMS 1 may provide a capability for viewing population wide data for the clinic or for a medical device registry in order to look at overall outcomes or relationships of patient characteristics.

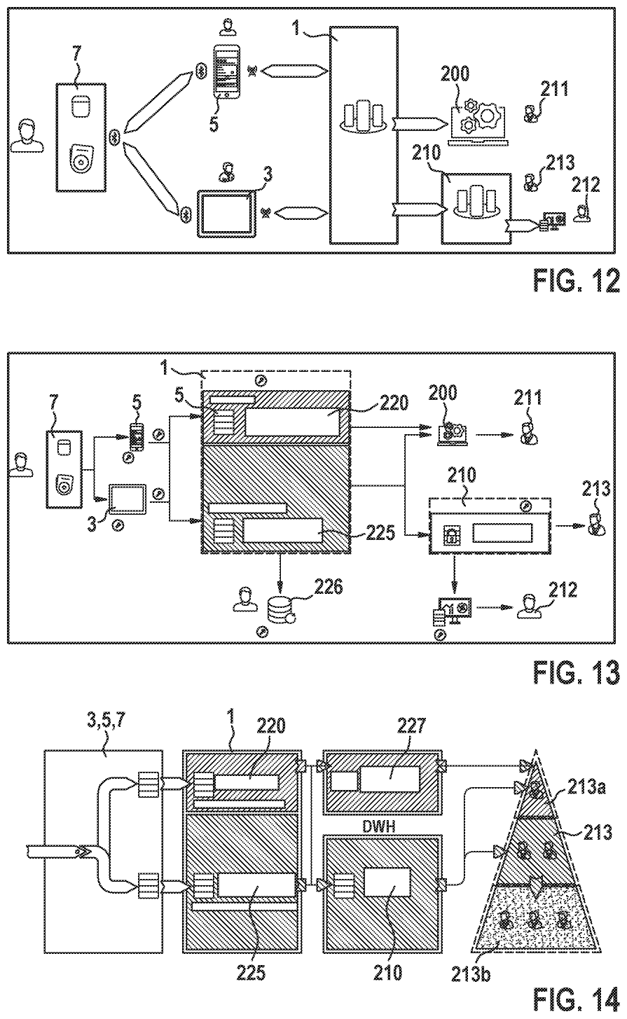

FIGS. 12 to 14 show different embodiments of data and access organization of the system. FIG. 12 shows that the RMS 1 data are accessible for customer service tools 200 and for a data warehouse (DWH) 210. The DWH 210 is defined as data consumer, whereas the customer service tools 200 function as data consumer and data reporter. The customer service tools 200 are used to manage the RMS 1 and to fix problems on the hardware or software side by a technician 211 of the service provider. As indicated above, some of the data available in the RMS 1 may be provided to the DWH 210 for, for example, a post market surveillance or the respectively medical devices. A report for said post marked surveillance may be defined by the AI module 2 as a report template. These data may be available for study managers 212 or employees of the medical device manufacturer 213. The systems shown in FIGS. 12 and 13 further provide direct access of the CP 3 to the medical device 7. This may be helpful for in person follow-up the clinic.

FIGS. 13 and 14 show that the data stored in the RMS 1 may be associated with the different security level categories. This may be controlled by the AI module 2 during definition of the data imports and data exports. One part of the data may be private or personal data 220 which need a higher level of protection than the pseudonymized data 225. The private or personal data 220 comprise, for example, names, addresses of patients, HCP or other users, patient record IDs, user IDs. The pseudonymized data 225 may comprise medical data, technical data, serial numbers, patient record IDs, user IDs. As one may derive from FIG. 13 the data warehouse 210 is allowed to access the pseudonymized data 225 only, whereas the customer service tools 200 need to have access to both, personal data 220 and pseudonymized data 225. Further, all data are stored in a backup storage 226.

In FIG. 14 it is shown that the employees of the medical device manufacturer may have different access rights. A first category of employees 213a may have access to personal data 220 via an interim storage 227 and pseudonymized data 225 via the DWH 210. The interim storage is defined by the AI module 2 as another data consumer. The access to the interim storage 227 is defined such that it is highly restricted. After finishing the tasks using the data of the interim storage 227 these data are deleted. The tasks of this group of employees may comprise contacting users for regulatory reasons or due to discovered issues with specific medical devices or troubleshooting related to personal data 220. The second group and third group of employees 213, 213b have access to pseudonymized data 225, only. The second and second largest group of employees 213 may provide, for example, troubleshooting related to specific medical devices or monitoring of specific medical devices (post market surveillance). The third and largest group of employees 213b provides, for example, statistical monitoring of all medical devices assigned to the system, forward analysis or troubleshooting without the need to access identifiers.

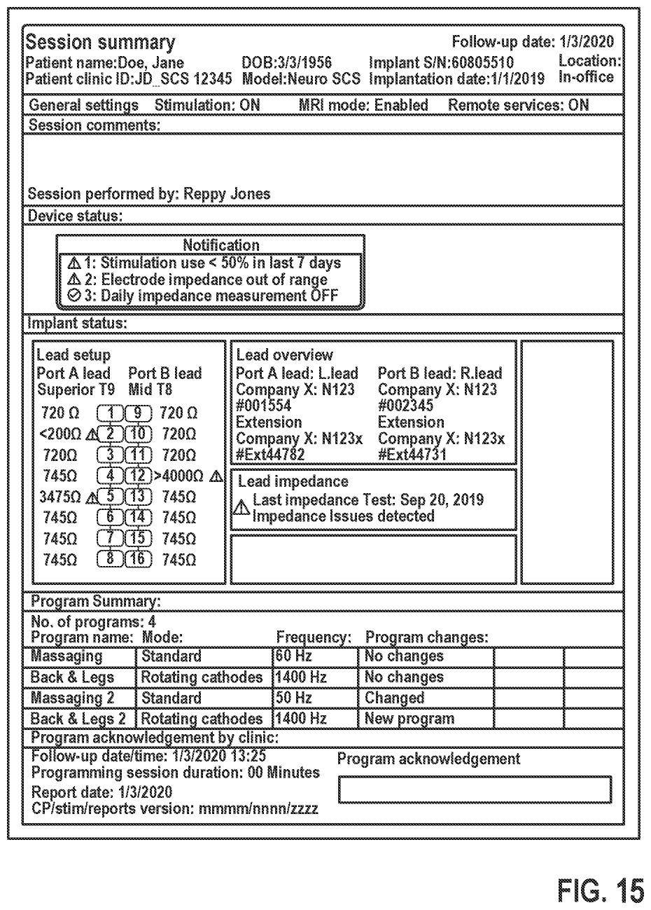

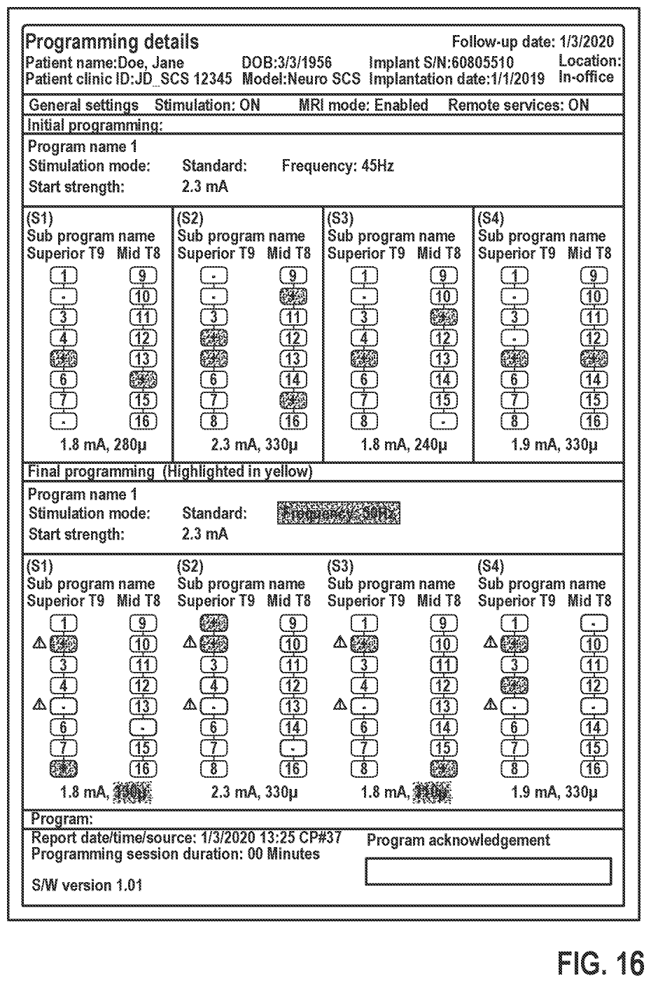

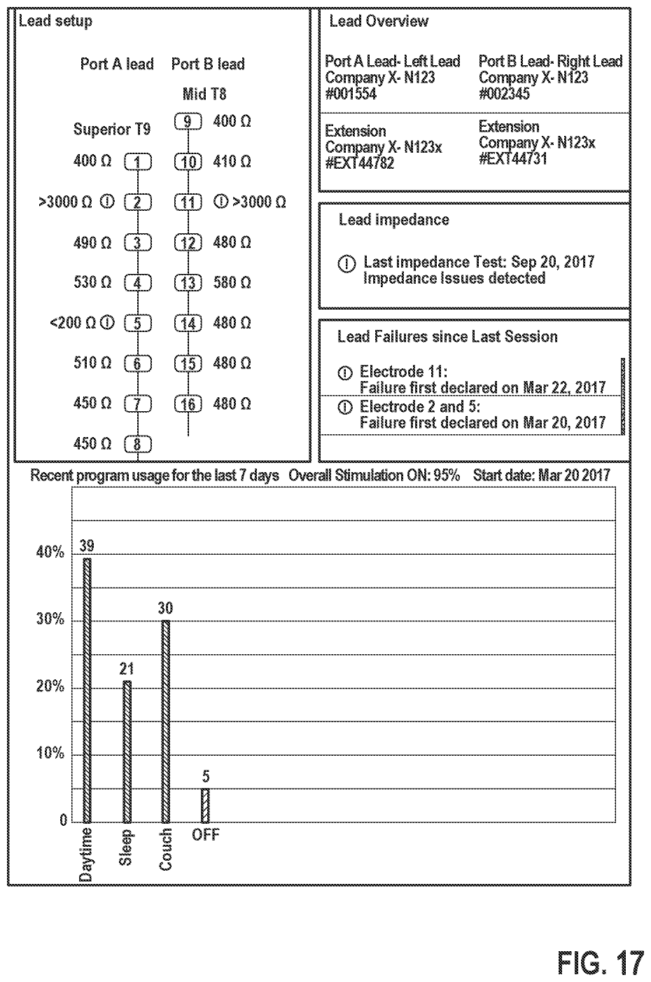

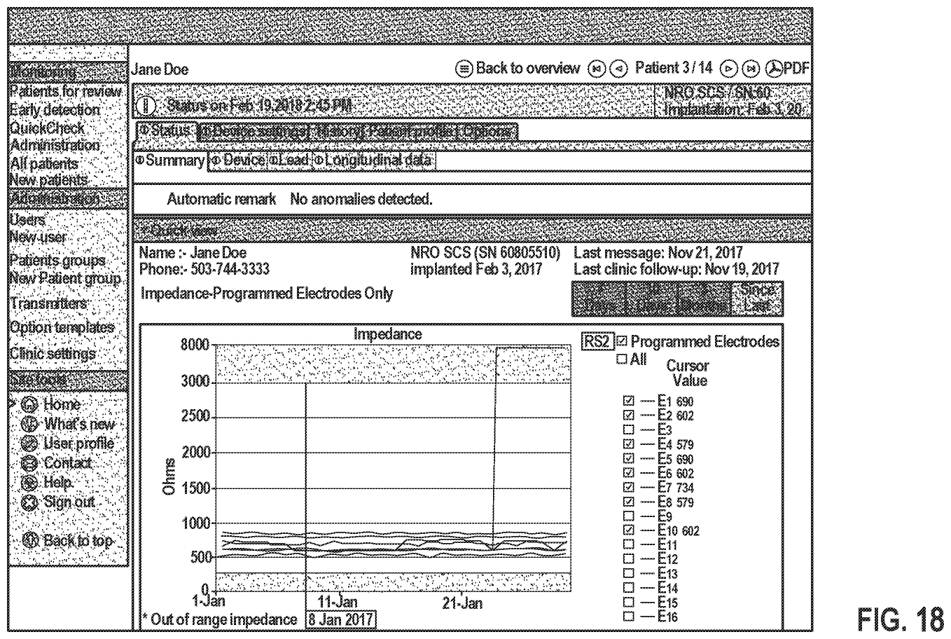

Regardless of which UI the user may accesses patient data and/or the remote programming process through a common print application which used for generation of medical device report(s). These reports consolidate current and historical, as well as objective and subjective data, into the single report. The report uses multiple display types: text fields, tabular data and data graphs. Examples for such reports are shown in FIGS. 15 to 18. These individual reports were created using the AI module 2 by arranging presentation objects in a data report template. The template was then stored in the central data repository 21.

This process facilitates tracking or “visualization” of the reprogramming process, describing the status of the reprogramming as it occurs, up to an indication of the successful completion of the reprogramming and annotation of the patient record with the updated status, in real-time via continuous interrogation and with data processing using AI algorithms.

It will be apparent to those skilled in the art that numerous modifications and variations of the described examples and embodiments are possible in light of the above teachings of the disclosure. The disclosed examples and embodiments are presented for purposes of illustration only. Other alternate embodiments may include some or all of the features disclosed herein. Therefore, it is the intent to cover all such modifications and alternate embodiments as may come within the true scope of this invention, which is to be given the full breadth thereof. Additionally, the disclosure of a range of values is a disclosure of every numerical value within that range, including the end points.

1. A remote monitoring server (RMS) configured to be used in a system comprising at least one medical device and at least one remote device of the group comprising at least one patient remote device and at least one health care professional (HCP) remote device, wherein the RMS comprises a communication module configured to communicate with the at least one remote device, wherein the RMS comprises a central data repository and at least one processor, wherein the central data repository is configured to store data collected by the at least one medical device and the at least one remote device, wherein the RMS further comprises an artificial intelligence (AI) module integrated within the at least one processor and/or cooperating with the at least one processor, wherein the AI module is configured to process data stored in the central data repository and/or to control data communication using the communication module and/or to provide services of a search engine.

2. The RMS of claim 1, wherein processing of data provided by the AI module comprises at least one operation of the operations comprising indexing of data, comparison of data, creating an envelope for received data, display of data, and managing data reports.

3. The RMS of any of claim 1, wherein controlling of data communication provided by the AI module using the at least one communication module comprises receiving and/or accepting data imports and/or creating and/or sending data exports from/for different data management systems, defining and/or approving at least one external data reporter and/or at least one external data consumer, defining, testing and/or controlling of at least one communication path.

4. The RMS of claim 1, wherein managing of data reports provided by the AI module comprises creating presentation objects, arranging presentation objects in a data report template, creating and storing a data report template, executing of a presentation object or a data report template thereby creating an individual data report and/or exporting data report templates for use in a remote device.

5. The RMS of claim 1, wherein services of a search engine provided by the AI module comprise, beside the indexing of data stored in the central data repository and, if applicable, of data stored in the at least one approved external data reporter, serving and ranking of search results after receiving a search query.

6. The RMS of claim 1, wherein the AI module is configured to provide AI submodules for use in the at least one remote device.

7. A system comprising an RMS of claim 1, at least one medical device and at least one remote device of the group comprising at least one patient remote device and at least one health care professional (HCP) remote device.

8. The system of claim 7, wherein at least one PR and/or the at least one CP comprises a software subscription module configured to use AI.

9. A method for operating a remote monitoring server (RMS) configured to be used in a system comprising at least one medical device and at least one remote device of the group comprising at least one patient remote device and at least one health care professional remote device, wherein the RMS comprises a communication module configured to communicate with the at least one remote device, wherein the RMS comprises a central data repository and at least one processor, wherein the central data repository stores data collected by the at least one medical device and the at least one remote device, wherein the RMS further comprises an artificial intelligence (AI) module which processes data stored in the central data repository and/or controls data communication using the communication module and/or provides services of a search engine.

10. The method of claim 9, wherein processing of data provided by the AI module comprises at least one operation of the operations comprising indexing of data, comparison of data, creating an envelope for received data, display of data, and managing data reports.

11. The method of claim 9, wherein controlling of data communication provided by the AI module using the at least one communication module comprises receiving and/or accepting data imports and/or creating and/or sending data exports from/for different data management systems, defining and/or approving at least one external data reporter and/or at least one external data consumer, defining, testing and/or controlling of at least one communication path.

12. The method of claim 9, wherein managing of data reports provided by the AI module comprises creating presentation objects, arranging presentation objects in a data report template, creating and storing a data report template, executing of a presentation object or a data report template thereby creating an individual data report and/or exporting data report templates for use in a remote device.

13. The method of 9, wherein services of a search engine provided by the AI module comprises, beside the indexing of data stored in the central data repository and, if applicable, of data stored in the at least one approved external data reporter, serving and ranking of search results after receiving a search query.

14. A computer program product comprising instructions which, when executed by at least one processing unit comprising the AI module or cooperating with the AI module, cause the at least one processing unit and/or AI module to perform the steps of the method according to claim 9.

15. Computer readable data carrier storing a computer program product according to claim 14.
